(19) United States
(12) Patent Application Publication
(10) Pub. No.: US 2026/0060937 A1
(43) Pub. Date: Mar. 5, 2026

(54) LIDOCAINE NON-AQUEOUS PATCH, 1.8% FOR PAIN ASSOCIATED WITH POST-HERPETIC NEURALGIA

(71) Applicant: Stira Pharmaceuticals LLC, Fairfield, NJ (US)

(72) Inventors: Satyanarayana VALIVETI, Fairfield, NJ (US); Surya NALLURI, Fairfield, NJ (US); Phani Prasanth TIRUMALASETTY, Fairfield, NJ (US)

(21) Appl. No.: 19/302,413

(22) Filed: Aug. 18, 2025

Related U.S. Application Data
(60) Provisional application No. 63/687,610, filed on Aug. 27, 2024.

(51) Int. Cl. A61K 9/70; A61K 31/167; A61K 47/02; A61K 47/14; A61P 25/04

(52) U.S. Cl. CPC A61K 9/7053; A61K 31/167; A61K 47/02; A61K 47/14; A61P 25/04

(57) ABSTRACT

The present invention relates to a lidocaine non-aqueous topical patch composition comprising approximately 1.8% w/w lidocaine incorporated into a non-aqueous adhesive matrix containing high and low molecular weight polyisobutylenes, hydrogenated polybutene, styrene-isoprene-styrene block copolymer, mineral oil, isopropyl myristate, silicon dioxide, and optionally antioxidants. The formulation provides enhanced skin permeation and sustained drug release with minimal crystallization. The patch is manufactured using a solvent-coating process with toluene, followed by drying and lamination to achieve desired coat weight and adhesive properties. The patch is effective for treating pain conditions such as post-herpetic neuralgia and offers improved stability, ease of application, and reduced risk of irritation.

### CROSS REFERENCE TO RELATED APPLICATIONS

The present application claims priority to U.S. Provisional Patent Application No. 63/687,610 filed on Aug. 27, 2024, which is hereby incorporated by reference in its entirety.

### FIELD OF THE INVENTION

The present invention relates to a lidocaine-containing topical non-aqueous patch for the treatment of localized pain conditions. More specifically, the invention pertains to a stable, adhesive, and sustained-release formulation of lidocaine using optimized combinations of polymers and permeation enhancers for controlled dermal delivery, reduced crystallization, and improved patient compliance.

### BACKGROUND OF THE INVENTION

Lidocaine, a common local anaesthetic, is available in various external preparations like ointments, creams, sprays, and patches. Aqueous base patches, such as Lidoderm, are used for postherpetic neuralgia but have drawbacks like poor skin compatibility, weak adhesion, and moisture vaporization issues. Non-aqueous patches, detailed in prior art, often contain high concentrations of lidocaine, which can lead to side effects and poor skin permeability due to the crystalline state of lidocaine. Techniques using lower lidocaine concentrations face challenges in maintaining long-term drug release and skin permeation. Additionally, other patents discuss using antiphlogistic analgesics in patches but do not address lidocaine specifically. Prior art also discloses Lidocaine non-aqueous patches comprising specific concentrations of Lidocaine and dissolving agents, such as organic acids and poly-alcohols, which influence drug release and absorption.

### SUMMARY OF THE INVENTION

In one aspect, the present invention provides a non-aqueous topical patch comprising lidocaine in a concentration of approximately 1.8% w/w within a carefully optimized adhesive matrix. The formulation includes high molecular weight polyisobutylene, low molecular weight polyisobutylene, hydrogenated polybutene, styrene-isoprene-styrene (SIS) block copolymer, mineral oil, isopropyl myristate, silicon dioxide, and optionally antioxidants like butylated hydroxytoluene (BHT).

In another aspect, the invention provides a patch wherein the synergistic combination of mineral oil and isopropyl myristate modulates the solubility and thermodynamic activity of lidocaine, facilitating optimal permeation through the skin while minimizing crystallization risks.

In yet another aspect, the invention discloses a manufacturing method for the non-aqueous lidocaine patch involving solvent-based processing, where ingredients are mixed in an organic solvent such as toluene, followed by coating, drying, and lamination steps to achieve a consistent coat weight and adhesive performance.

Another aspect of the invention includes a method for treating pain associated with post-herpetic neuralgia by applying the patch to the affected area, thereby providing sustained analgesic effects without significant skin irritation.

The invention further relates to a product produced by a solvent coating process, wherein a homogeneous drug-in-adhesive matrix is achieved through sequential addition of excipients in toluene, followed by lamination with a non-woven fabric backing.

The present invention achieves controlled lidocaine release, typically over 6% within 12 hours of application, while ensuring formulation stability and minimizing skin irritation.

### BRIEF DESCRIPTION OF THE DRAWINGS

FIG. 1 depicts the influence of mineral oil concentration on the cumulative release of lidocaine from the non-aqueous patch formulations, highlighting how increasing the concentration of mineral oil as a tackifier improves the adhesive properties of the patch, enabling greater mobility of Lidocaine within the matrix. This enhanced mobility facilitates the formation of diffusion channels in the matrix, resulting in a higher drug release.

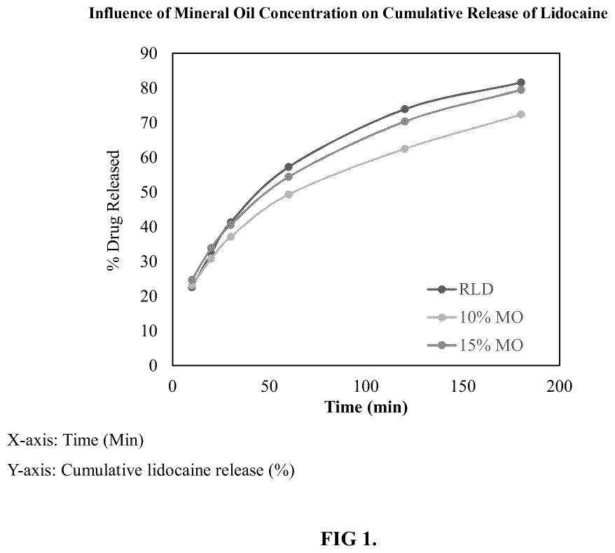

FIG. 2 illustrates the effect of varying ratios of isopropyl myristate (IPM) and mineral oil (MO) on the cumulative amount of lidocaine permeated (expressed in μg/cm²) across human cadaver skin using in-vitro permeation testing (IVPT), demonstrating the role of excipient balance in optimizing drug release.

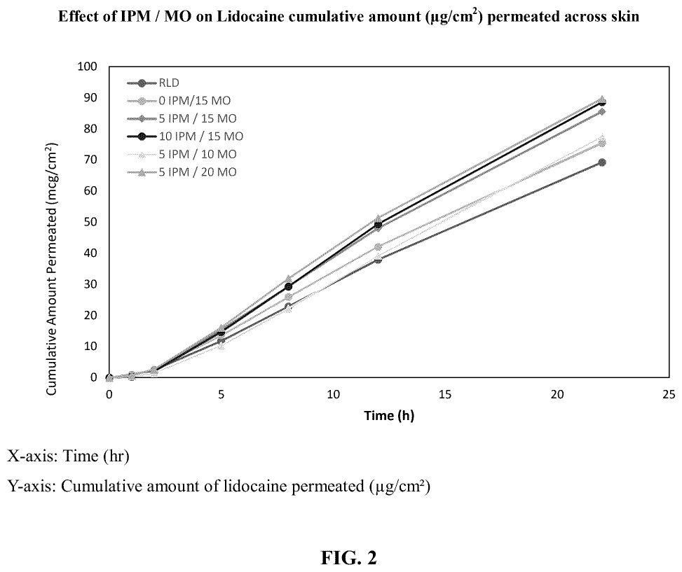

FIG. 3 shows the effect of different ratios of isopropyl myristate (IPM) and mineral oil (MO) on the average flux of lidocaine (expressed in μg/cm²/hour), highlighting how excipient composition impacts the rate of Topical drug delivery system.

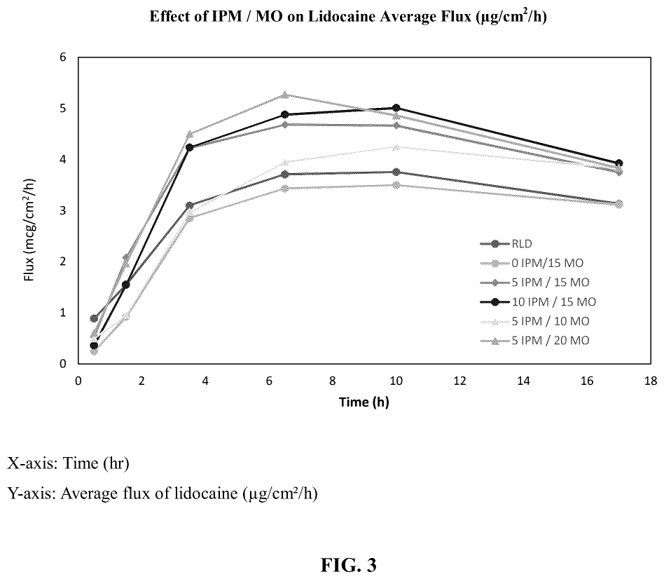

### DETAILED DESCRIPTION

For the purposes of the present invention, the following terms are defined along with their corresponding methods of measurement, unless specified otherwise by the context:
“Lidocaine” refers to 2-(diethylamino)-N-(2,6-dimethylphenyl) acetamide, a local anaesthetic used for relieving pain. The quantification of Lidocaine in the formulation is performed using a validated High-Performance Liquid Chromatography (HPLC) method, in accordance with pharmacopeial standards or ICH guidelines.
“Non-aqueous patch” means a Topical drug delivery system or Topical patch formulation that excludes water as a solvent. It consists of lipophilic excipients, polymers, and active pharmaceutical ingredients, designed for sustained delivery of the drug through the skin.
“High Molecular Weight Polyisobutylene (HMW PIB)” is polyisobutylene with a weight-average molecular weight (Mw) typically greater than 500,000 Daltons, such as Oppanol N100. The molecular weight is confirmed using Gel Permeation Chromatography (GPC).
“Low Molecular Weight Polyisobutylene (LMW PIB)” denotes polyisobutylene with Mw typically less than 100,000 Daltons, such as Oppanol B12 or N80. GPC is employed for its molecular weight determination.
“Polybutene” refers to hydrogenated polybutene, such as Panalane H-300, functioning as a tackifier and viscosity modifier in the formulation. Its molecular weight is measured by GPC and its purity by Gas Chromatography (GC).
“Styrene-Isoprene-Styrene (SIS) Block Copolymer” is a triblock copolymer that imparts cohesive strength and elasticity to the adhesive matrix. Its chemical composition is confirmed via Nuclear Magnetic Resonance (NMR), and molecular weight by GPC.
“Mineral Oil (MO)” is a pharmaceutically acceptable highly refined liquid paraffin, used for its plasticizing and release-modifying properties. Its identity and purity are confirmed via GC.
“Isopropyl Myristate (IPM)” refers to an ester of isopropanol and myristic acid used as a permeation enhancer. It is quantified and identified using HPLC or GC.
“Silicon Dioxide” refers to colloidal silicon dioxide used as a Matrix modifier. It is characterized through Loss on Drying (LOD), X-Ray Diffraction (XRD), and particle size distribution analysis.
“Butylated Hydroxytoluene (BHT)” denotes an antioxidant used to prevent oxidative degradation in the formulation. BHT is quantified using HPLC or GC.
“Drug-in-Adhesive (DIA)” refers to a system where the drug is homogeneously dispersed or partially solubilized within the adhesive matrix. Uniformity is verified using HPLC and optical microscopy.
“Solvent coating process” involves the dissolution or dispersion of all formulation components in an organic solvent, followed by coating on a backing layer and drying. Residual solvents are measured per ICH Q3C guidelines using GC.
“Coat weight” is the mass per unit area (mg/cm²) of the dried adhesive layer, determined gravimetrically using a precision analytical balance.
“Target coat weight” is the predetermined mass per unit area required to deliver the intended drug dose, validated via direct weighing post-manufacture.
“Polyethylene Terephthalate (PET) Film” is a polymeric substrate acting as the release liner or backing, with thickness measured using micrometre calipers.
“Non-woven fabric” refers to a breathable support layer with basis weight measured by GSM (grams per square meter) method.
“Release liner” is a protective layer to prevent premature adhesion, characterized using peel adhesion testing as per ASTM D3330.
“Thermodynamic activity” denotes the driving force for diffusion of the drug from the patch, inferred from solubility and IVPT (In-Vitro Permeation Test) data, and quantified via HPLC.
“Flux” is the permeation rate (μg/cm²/hour), derived from the linear portion of cumulative release profiles obtained in IVPT using Franz diffusion cells.
“In-Vitro Permeation Test (IVPT)” uses Franz diffusion cells with human cadaver or animal skin as the membrane, and cumulative permeation is quantified via HPLC.
“Cumulative drug release” denotes the total drug released per unit area (e.g., μg/cm²) over a specified duration, measured through IVPT.
“Crystallization” refers to drug crystal formation in the adhesive matrix, evaluated using Polarized Light Microscopy (PLM) and XRD.
“Storage Conditions” follow ICH Q1A (R2): refrigerated (4° C.), room temperature (25° C./60% RH), and accelerated (40° C./75% RH).
“Release Ratio” is the percent of total drug released from the patch after a fixed period (e.g., 12 hours), calculated using IVPT data.
“Post-Herpetic Neuralgia (PHN)” refers to nerve pain following herpes zoster, where sustained release formulations offer therapeutic benefits.
The term “tackifier” as used herein refers to materials other than polyisobutylene (PIB) that are added to adhesives to increase their tack or stickiness. These materials are typically naturally occurring resinous or rosinous substances or truly synthetic polymer materials. An adhesive is considered substantially free of tackifier if it contains, at most, trace amounts of tackifier, and preferably none.
“Bioavailability” refers to the extent and rate at which lidocaine enters systemic circulation when administered through the patch, inferred through IVPT cumulative release and flux data.
“Long-term storage” refers to conditions of 25° C./60% RH for at least six months as per ICH Q1A guidelines.
“Accelerated storage” refers to storage at 40° C./75% RH for three months per ICH Q1A.
“Flux improvement” means an increase of at least 10-20% in microgram/cm²/hour drug permeation compared to reference products, validated through IVPT.

Aqueous-based lidocaine preparations have poor adhesive properties and tend to fall off easily. Additionally, these preparations have less than 5% bioavailability of the lidocaine they contain. Lidocaine's basic ingredients are readily soluble in organic solvents like methanol, ethanol, and diethyl ether, but they are not easily dissolved in water.

The art of drug-releasing characteristics for aqueous-based patches has not been well-formulated. Practical considerations such as the type of support used for the preparation, ensuring stable attachment to the skin, and maintaining favorable release of the active ingredient have not been adequately addressed.

When lidocaine is formulated into non-aqueous patches, the adhesive power of the preparation tends to decrease as the lidocaine content increases. This is because lidocaine has partial compatibility with lipid-soluble tackifier resins, which lowers the adhesion properties of the resin. It is common practice to dissolve lidocaine in a solvent to formulate it into a patch and ensure effective release into the skin. However, higher amounts of solvent significantly reduce adhesive power.

The purpose of the present invention is to develop a non-aqueous base used in patches that can relieve muscle pain for extended periods. The invention utilizes a small amount of lidocaine, instead of high concentrations, in a patch that ensures stable and prolonged Topical absorption of lidocaine into the muscle.

The current invention utilizes adhesives, matrix modifiers, tackifiers, permeation enhancers, solubilizers, and, if necessary, antioxidants.

The present invention utilizes a biaxially-oriented stretch cloth as the support for the non-aqueous patch. Once applied to the skin, the patch follows the skin's expansion and contraction, making it unlikely to fall off. The invention also regulates the adhesive power to ensure that the patch adheres securely to the skin and provides long-lasting and effective Topical absorption.

The biaxially-oriented stretch cloth used in this invention has a stretch strength of less than 2000 g/50 mm for 50% longitudinal extension (testing sample size: width 50 mm, length 300 mm, testing length 200 mm, tension rate 500 mm/min).

The appropriate adhesive power, measured using the 180-degree peel test defined in JIS (Japanese Industrial Standards) Z0237, ranges from 0.4N/25 mm to 5N/25 mm. If the adhesive power exceeds 5N/25 mm, it may cause skin injury and contact dermatitis due to excessive tension. Conversely, if the adhesive power is below 0.4N/25 mm, the patch may easily detach, especially when the skin sweats, preventing the preparation from being maintained on the skin.

To achieve the desired adhesive properties and compatibility with lidocaine, the patch uses rubber-based materials such as polyisoprene rubber, polyisobutylene, styrene-isoprene rubber, and styrene-butadiene rubber. A combination of polyisobutylene (HMW+LMW), polybutene, and styrene-isoprene-styrene block copolymer (SIS) is particularly effective in maintaining the patch appropriate firmness and cohesiveness to stick on the skin. The rubber-based material typically constitutes 10 to 50 mass percent of the patch, preferably 20 to 40 mass percent. The amount of SIS between 20-25% is critical for the formulation.

The adhesive power is controlled using tackifier. The adhesive composition of this invention contains both HMW PIB and LMW PIB. HMW PIB concentration ranges from 5-15%, whereas LMW PIB ranges from 20-35%.

It is preferable to minimize or eliminate extraneous components in the adhesive mixture to reduce the potential for irritation or allergic reactions when the Topical delivery system contacts the skin. However, dyes, pigments, inert fillers, stabilizers, matrix modifiers such as colloidal silicon dioxide (which provides fluid-filled channels for drug release), or other additives (excluding plasticizers and tackifiers) well-known to the art may be added if desired.

As a softener, polybutene or mineral oil may be added. When styrene-isoprene-styrene block copolymer is used, adding mineral oil results in a good patch by enhancing the elastic force of the patch. The amount of mineral oil is less than the amount of styrene-isoprene-styrene block copolymer. The combination helps reduce the tackiness of PIBs.

To optimize the formulation, additives like silicon dioxide, zinc oxide, and zinc stearate may be incorporated to regulate the release and stability of lidocaine. Silicon dioxide is included as a matrix modifier to assist in the retention of isopropyl myristate and mineral oil and to create liquid-filled channels that facilitate drug release.

Lidocaine may be in a crystalline state, which results in a lower release rate and less effective use. Therefore, it is preferable to include lidocaine in a dissolved state using effective dissolving agents.

According to Fick's law, drug release depends on the amount of unbound or free dissolved drug in the matrix available for diffusion, also known as thermodynamic activity. Patch area activity (% cm−2), a surrogate measurement of thermodynamic activity (EMEA, 2012), measures the formulation's intrinsic capability to release drug substance from the patch in vivo and is estimated by drug utilization rate per patch size. This thermodynamic activity acts as a driving force for increasing the absorption of drugs by the skin and can be modulated by the drug's physicochemical parameters, such as solubility, partition coefficient, and water activity within the vehicle. The state of the drug matrix—whether it is subsaturated, saturated, or supersaturated—can significantly impact drug release. For optimum drug release, it is essential that the drug is above saturation and maintained in a dissolved state.

In the present invention, lidocaine loading in the drug matrix is 36 mg, constituting 1.8% of the formulation. Polymers laminated with 1.0%, 1.8%, 3.0%, and 5.0% lidocaine and stored under various conditions (25° C./60% RH, 40° C./75% RH, and 4° C. at 2 weeks) did not exhibit lidocaine crystals. This indicates that at 1.8%, lidocaine is present at a saturated level in the matrix, resulting in low thermodynamic activity. Low thermodynamic activity implies that the drug has a lower tendency to move out of the delivery system into the surrounding environment. Increasing the thermodynamic activity of a drug can be achieved by making the system supersaturated, either by increasing drug loading or incorporating excipients that have low solubility for the drug in the matrix. However, this approach has the major limitation of formulation instability, which can compromise efficacy. Therefore, the right combination of excipients must be used to increase the thermodynamic activity of the drug in the formulation while stabilizing the formulation. In the present invention, a combination of mineral oil and isopropyl myristate is used to improve the thermodynamic activity of lidocaine in the patch.

The adhesive properties of a Topical delivery system, such as tack and peel, are influenced by the choice of excipients and the manufacturing process (solvent casting vs. extrusion). The choice of process solvent significantly impacts adhesive properties. For this formulation, heptane and toluene were used to dissolve high and low molecular weight polyisobutylene (PIB). The formulation made with PIB in heptane exhibited higher tack, making it unsuitable for wear, especially considering the indication of the lidocaine non-aqueous patch (1.8%)—relief of pain associated with post-herpetic neuralgia. In contrast, toluene provided an adhesive with the right balance of cohesive strength and adhesive strength, resulting in optimum adhesive properties suitable for the indication. Hence, toluene is critical as a process solvent in the current composition.

Dissolving agents and permeation enhancers increase drug permeability across the skin by temporarily altering or disrupting the skin barrier function. They achieve this by disrupting the stratum corneum, modifying lipid bilayers, increasing skin hydration, and interfering with keratinization. The use of such excipients in topical formulations, alone or in combination, might lead to skin irritation, especially in pre-existing skin conditions like neuralgia. Considering the patch's indication (neuralgia associated with shingles, where the affected area is very sensitive), reducing or eliminating the use of dissolving agents and permeation enhancers could provide a significant advantage over the innovator patch.

The combination of mineral oil and low concentration of isopropyl myristate serves as a permeation enhancer and solubilizer for lidocaine, improving drug release and absorption through the skin. It ensures that lidocaine stays partially dissolved without crystallizing and effectively relieves muscle pain over long periods.

The concentration of isopropyl myristate between 5% and 10% helps dissolve lidocaine completely. Though IPM is a preferred choice, other fatty acids such as oleic acid, stearic acid, etc., as alternatives for IPM can also be used.

The concentration of mineral oil between 5% and 15% reduces the solubility of lidocaine and promotes increased drug release.

The reduced solubility of lidocaine in mineral oil enhances its thermodynamic activity, leading to a higher rate of drug release. This is because the addition of mineral oil increases the amount of free drug available for release, as lidocaine is less soluble in the formulation matrix. Consequently, formulations with higher mineral oil content exhibit greater drug release compared to those with lower mineral oil content.

The ratio of isopropyl myristate to mineral oil is adjusted such that the formulation remains clear at a concentration of lidocaine of 1.8%, with no suspended lidocaine particles. The most preferred ratio of isopropyl myristate to mineral oil is between 1:2 and 1:3.

The drug release rate is higher in formulations containing 15% mineral oil compared to those with 10% mineral oil. A mineral oil concentration between 10-15% is preferred for the formulation.

The formulation remains stable and free of crystallization of lidocaine under various storage conditions for at least four weeks.

The lidocaine concentration in the patch typically ranges from 0.5 to 7 mass percent. Lidocaine is used at 1.8% in the current formulation.

The patch uses a biaxially-oriented stretch cloth substrate with a peel strength of 0.4N/25 mm to 5N/25 mm, ensuring long-term adhesion and effective lidocaine release without damaging the skin. Good lidocaine release means achieving more than a 6% release ratio, measured by the difference between the remaining lidocaine after 12 hours on the skin and the initial amount in the preparation.

The patch is produced using general methods such as solvent coating. The mass of the patch typically ranges from 60 to 200 g/m², with 80 to 180 g/m² being preferable. Below 60 g/m², lidocaine crystallization and poor adhesion occur, while above 200 g/m², the patch becomes too heavy and prone to dripping. The target mass is 142.8 g/m².

In one embodiment of the present invention, there is provided a lidocaine non-aqueous patch comprising 1.8% lidocaine by weight in a non-aqueous base, together with high molecular weight polyisobutylene, low molecular weight polyisobutylene, polybutene, styrene/isoprene/styrene (SIS) block copolymer, mineral oil, silicon dioxide, and isopropyl myristate.

In another embodiment of the present invention, the lidocaine non-aqueous patch comprises lidocaine at 1.8% weight by weight, equivalent to approximately 36 mg in the drug matrix.

In yet another embodiment of the present invention, the lidocaine non-aqueous patch is characterized by a lidocaine release ratio of more than 6% after 12 hours of attachment to the skin, providing sustained drug delivery during application.

In another embodiment of the present invention, the amount of lidocaine incorporated in the patch ranges from 0.1 mg/cm² to 1 mg/cm² of the patch, enabling precise dosage delivery per unit area.

In one embodiment of the present invention, the concentration of styrene/isoprene/styrene block copolymer within the patch is between 20% and 25% by weight, optimizing both the mechanical strength and the adhesion performance of the patch.

In a further embodiment of the present invention, the isopropyl myristate concentration ranges between 5% and 10% by weight, effectively balancing drug solubility, release, and skin permeation while minimizing irritation.

In another embodiment of the present invention, the concentration of mineral oil ranges from 5% to 15% by weight, with a preferred range between 10% and 15%, to modulate the drug's thermodynamic activity and adhesive characteristics.

In another embodiment of the present invention, the ratio of isopropyl myristate to mineral oil is maintained between 1:2 and 1:3, ensuring appropriate drug solubility for optimized release profile from the adhesive matrix.

In another embodiment of the present invention, the combination of mineral oil and isopropyl myristate acts as a permeation enhancer and solubilizer for lidocaine, thereby enhancing drug release and skin absorption without compromising formulation stability.

In another embodiment of the present invention, the lidocaine non-aqueous patch includes optional excipients such as zinc oxide, zinc stearate, and antioxidants, which may provide added skin protection, oxidative stability, or anti-inflammatory benefits.

In a further embodiment of the present invention, the role of mineral oil and isopropyl myristate in modulating lidocaine's skin permeation behavior is investigated using in-vitro permeation testing (IVPT) with human cadaver skin, highlighting their importance in optimizing drug release and permeation profiles.

In another embodiment of the present invention, the lidocaine non-aqueous patch is prepared by a solvent coating process, wherein all excipients are dissolved or dispersed in an organic solvent followed by coating onto a backing layer and subsequent drying to achieve the desired coat weight.

In another embodiment of the present invention, the selected process solvent is toluene, which provides balanced cohesive and adhesive properties to the drug-in-adhesive matrix, contributing to optimal patch performance during wear, particularly for indications such as post-herpetic neuralgia.

In a further embodiment of the present invention, the formulation process involves mixing all excipients in toluene followed by coating and drying steps to obtain the target coat weight and uniformity across the patch surface.

In another embodiment of the present invention, the lidocaine non-aqueous patch is manufactured by sequentially adding styrene-isoprene-styrene block copolymer, high and low molecular weight polyisobutylene, polybutene, silicon dioxide, BHT, mineral oil, isopropyl myristate, and lidocaine into toluene to form a homogeneous drug-in-adhesive solution, which is then coated onto a polyester film.

In one embodiment of the present invention, the preparation of the lidocaine non-aqueous patch includes dispensing toluene into a mixing vessel, followed by the sequential addition of colloidal silicon dioxide, mineral oil, isopropyl myristate, and lidocaine with thorough mixing to achieve dissolution and uniform dispersion. Subsequently, SIS block copolymer is added and solvated completely, followed by addition of polyisobutylene and polybutene with continued mixing until a uniform mixture is obtained.

In another embodiment of the present invention, the solid content of the adhesive mixture is carefully controlled to maintain the desired coat weight and uniformity upon drying, critical for ensuring consistent drug content and release performance.

In yet another embodiment of the present invention, the drug-in-adhesive mixture is coated onto a PET film and dried to reach the desired target coat weight, followed by lamination with a non-woven fabric to enhance flexibility and comfort.

In another embodiment of the present invention, a second layer of drug-in-adhesive mixture is coated onto a PET liner to achieve the same target coat weight, and this layer is laminated with the previously coated non-woven fabric, forming a composite structure with the adhesive matrix sandwiched between the non-woven fabric and PET release liner.

In a preferred embodiment of the present invention, the target coat weight is about 14.28 mg/cm², ensuring optimal drug delivery performance during patch application.

In another embodiment of the present invention, the patch formulation is free from visible lidocaine crystallization throughout its shelf-life, indicating excellent drug stability within the adhesive matrix.

In a further embodiment, the formulation remains free of crystallization and maintains potency within 90-110% of label claim over at least 3 months under ICH accelerated (40° C./75% RH) and long-term (25° C./60% RH) storage conditions.

In yet another embodiment, the patch may be used to treat localized pain, including but not limited to post-herpetic neuralgia, muscle pain, neuralgia of various origins, diabetic neuropathy, and localized joint pain.

In one embodiment of the present invention, the lidocaine non-aqueous patch is indicated for treating pain associated with post-herpetic neuralgia, wherein application of the patch to the affected area delivers sustained therapeutic pain relief without causing significant skin irritation.

In one embodiment of the present invention, the lidocaine non-aqueous patch comprises a drug-in-adhesive matrix configured to maintain lidocaine in a partially solubilized state by utilizing a specific combination of mineral oil and isopropyl myristate, thereby enhancing thermodynamic activity and promoting sustained drug release.

In one embodiment, the total mass of adhesive matrix is between 60 g/m² and 200 g/m², preferably between 80-180 g/m², more preferably between 135-145 g/m², ensuring optimal adhesion and drug delivery characteristics without risk of patch delamination or drug crystallization.

In one embodiment, the formulation demonstrates flux of lidocaine through human cadaver skin of at least 2.5 μg/cm²/hr, preferably 3-4 μg/cm²/hr, more preferably about 3.5 μg/cm²/hr, determined using IVPT methods described herein.

In one embodiment of the present invention, the lidocaine non-aqueous patch exhibits enhanced skin permeation with an average flux greater than 3 μg/cm²/h, as determined via in-vitro permeation testing using human cadaver skin, through the optimized use of mineral oil and isopropyl myristate as permeation enhancers.

In one embodiment of the present invention, the lidocaine non-aqueous patch maintains physical stability without crystallization under storage at 4° C., room temperature, and accelerated conditions, even at lidocaine concentrations up to 1.8% w/w.

In one embodiment of the present invention, the patch comprises a dual-layer coating system wherein two successive layers of drug-in-adhesive composition are coated and laminated to achieve improved uniformity, mechanical stability, and consistent drug release.

In one embodiment of the present invention, the lidocaine non-aqueous patch exhibits a higher cumulative release and superior flux profile compared to a reference lidocaine patch product, particularly when the ratio of mineral oil to isopropyl myristate is maintained between 2:1 to 3:1.

In one embodiment of the present invention, the patch is manufactured via a solvent coating process wherein the sequence of excipient addition and control of solid content are critical to achieving a homogenous, stable drug-in-adhesive matrix.

In another embodiment, the patch is packaged in a light-protective, low-permeability pouch comprising aluminum laminate or polyethylene with oxygen and moisture barrier properties, optionally with desiccant inclusion.

The lidocaine non-aqueous patch of the present invention may be packaged in a single-use or multi-use sealed pouch with low moisture vapor transmission rate (MVTR). The preferred packaging includes aluminum-polyester laminate pouches with integrated desiccant to maintain moisture levels below 10% RH during storage. Such packaging ensures product stability, reduces lidocaine degradation, and prevents premature crystallization during shelf life.

The patch may be packaged in a heat-sealed pouch comprising aluminum-polyethylene laminate with a water vapor transmission rate (WVTR) of less than 0.05 g/m²/day, further ensuring minimal moisture ingress during storage.

### Examples 1-3

| | Example 1 | Example 2 | Example 3 |
|---|---|---|---|
| Components | % w/w | | |
| Polyisobutene (Oppanol N100) | 6.3 | 12.6 | — |
| Polyisobutene (Oppanol N80) | — | — | 12.6 |
| Polyisobutene (Oppanol B12) | 31.6 | 25.3 | 25.3 |
| Hydrogenated Polybutene | 25.3 | 25.3 | 25.3 |
| (Panalane H-300) | | | |
| Styrene-isoprene-styrene block | 20.0 | 20.0 | 20.0 |
| copolymer | | | |
| Mineral oil | 10.0 | 10.0 | 10.0 |
| Isopropyl Myristate | 5.0 | 5.0 | 5.0 |
| Silicon dioxide | 1.0 | 1.0 | 1.0 |
| Lidocaine | 1.8 | 1.8 | 1.8 |

### Examples 4-9

| | Ex. 4 | Ex. 5 | Ex. 6 | Ex. 7 | Ex. 8 | Ex. 9 |
|---|---|---|---|---|---|---|
| Components | % w/w | | | | | |
| Polyisobutene (Oppanol | 14.4 | 12.4 | 11.4 | 10.4 | 10.4 | 10.4 |
| N100) | | | | | | |
| Polyisobutene (Oppanol B12) | 28.9 | 24.9 | 22.9 | 20.9 | 20.9 | 20.9 |
| Hydrogenated Polybutene | 28.9 | 24.9 | 22.9 | 20.9 | 20.9 | 20.9 |
| (Panalane H-300) | | | | | | |
| Styrene-isoprene-styrene | 10.0 | 20.0 | 25.0 | 25.0 | 25.0 | 25.0 |
| block copolymer | | | | | | |
| Mineral oil | 10.0 | 10.0 | 10.0 | 15.0 | 10.0 | 15.0 |
| Isopropyl Myristate | 5.0 | 5.0 | 5.0 | 5.0 | 5.0 | 5.0 |
| Silicon dioxide | 1.0 | 1.0 | 1.0 | 1.0 | 1.0 | 1.0 |
| Lidocaine | 1.8 | 1.8 | 1.8 | 1.8 | 1.8 | 1.8 |
| BHT | — | — | — | — | 0.02 | 0.02 |

### Process:

1. Dispensing Toluene: Begin by dispensing toluene into the mixing jar.
2. Adding Ingredients: Add colloidal silicon dioxide, mineral oil, IPM, and lidocaine into the mixing tank. Mix thoroughly to dissolve the lidocaine and disperse the silicon dioxide.
3. Incorporating SIS Polymer: Add the SIS polymer and mix until it is fully solvated.
4. Adding Oppanol and Panalane: Follow by adding Oppanol high molecular weight and mix until it is solvated, then add Oppanol B12 and Panalane H-300, mixing until a homogeneous mixture is achieved. The solid content of this mixture is 36%. The solid content of the drug in adhesive (DIA) is critical for achieving the desired coat weight.
5. Coating and Drying: The DIA is then coated onto a PET film and dried to reach a target coat weight of 7.14 mg/cm². The resulting laminate is then laminated with a non-woven fabric.
6. Second Layer Application: A second layer of DIA is coated onto the PET liner to achieve the same target coat weight of 7.14 mg/cm². As this second layer exits the drying tunnel, it is laminated with the previously coated non-woven fabric, effectively sandwiching the adhesive layer between the non-woven fabric and the PET release liner.

### Solubility Studies in Mineral Oil and IPM:

Solubility data indicating Lidocaine solubility in Mineral oil, IPM and combination of MO and IPM. Lidocaine is soluble in the concentration of IPM used in the formulation and has very solubility in Mineral oil. Adding Mineral oil to IPM reduces the solubility of Lidocaine in the composition which helps in improving the thermodynamic activity of the drug in the formulation. It is confirmed form the drug release shows higher drug release from 15% Mineral oil composition compared to 10% Mineral oil composition.

| S. | | |
|---|---|---|
| No. | Composition | Observation (24 hrs.) |
| 1 | 1.8% Lido + 5% IPM | Clear solution with few Lidocaine particles. |
| 2 | 1.8% Lido + 15% MO | Hazy mixture with suspended Lidocaine |
| | | particles. |
| 3 | 1.8% Lido + 10% MO + 5% IPM | Hazy mixture with suspended lidocaine |
| | | particles. |
| 4 | 1.8% Lido + 10% MO + 7.5% IPM | Clear solution with no Lidocaine particles |
| 5 | 1.8% Lido + 10% MO + 10% IPM | Clear solution with no Lidocaine particles |
| 6 | 1.8% Lido + 15% MO + 5% IPM | Hazy mixture with suspended lidocaine |
| | | particles. |
| 7 | 1.8% Lido + 15% MO + 7.5% IPM | Clear solution with no Lidocaine particles |
| 8 | 1.8% Lido + 15% MO + 10% IPM | Clear solution with no Lidocaine particles |

### Lidocaine Drug Release (Thermodynamic Activity):

Increasing the concentration of mineral oil as a tackifier improves the adhesive properties of the patch, enabling greater mobility of Lidocaine within the matrix. This enhanced mobility facilitates the formation of diffusion channels in the matrix, resulting in a higher drug release rate.

Lidocaine Crystal studies (Lidocaine + PIB + SIS)

| | 0 wk | | | 1 wk | | | 2 wk | | | 4 wk | | |
|---|---|---|---|---|---|---|---|---|---|---|---|---|
| Composition | 4° C. | CRT | ACC | 4° C. | CRT | ACC | 4° C. | CRT | ACC | 4° C. | CRT | ACC |
| 1.0% Lidocaine | x | x | x | x | x | x | x | x | x | x | x | x |
| 1.8% Lidocaine | x | x | x | x | x | x | x | x | x | x | x | x |
| 3.0% Lidocaine | x | x | x | x | x | x | x | x | x | x | x | x |
| 5.0% Lidocaine | x | x | x | x | x | x | x | x | x | x | x | x |

Lidocaine Crystal studies (Lidocaine + formulation components)

| | 0 wk | | | 1 wk | | | 2 wk | | | 4 wk | | |
|---|---|---|---|---|---|---|---|---|---|---|---|---|
| Composition | 4° C. | CRT | ACC | 4° C. | CRT | ACC | 4° C. | CRT | ACC | 4° C. | CRT | ACC |
| 1.4% Lidocaine | x | x | x | x | x | x | x | x | x | x | x | x |
| 1.8% Lidocaine | x | x | x | x | x | x | x | x | x | x | x | x |
| 2.3% Lidocaine | x | x | x | x | x | x | x | x | x | x | x | x |
| 5.0% Lidocaine | x | x | x | x | x | x | x | x | x | x | □ | x |

x = No crystal;
□ = Crystal

### Example 10

Effect of Mineral Oil Concentration

| | Example 1 | Example 2 |
|---|---|---|
| Components | (% w/w) | (% w/w) |
| Polyisobutene (Oppanol N80) | 12.44 | 10.44 |
| Polyisobutene (Oppanol B12) | 24.88 | 20.88 |
| Hydrogenated Polybutene (Panalane H-300) | 24.88 | 20.88 |
| Styrene-isoprene-styrene block copolymer | 20.00 | 20.00 |
| Mineral oil | 10.00 | 20.00 |
| Isopropyl myristate | 5.00 | 5.00 |
| Silicon dioxide | 1.00 | 1.00 |
| Lidocaine | 1.80 | 1.80 |

### Examples 11-13

Effect of Isopropyl Myristate Concentration

| | Example 3 | Example 4 | Example 5 |
|---|---|---|---|
| Components | (% w/w) | (% w/w) | (% w/w) |
| Polyisobutene (Oppanol N80) | 12.44 | 11.44 | 10.44 |
| Polyisobutene (Oppanol B12) | 24.88 | 22.88 | 20.88 |
| Hydrogenated Polybutene | 24.88 | 22.88 | 20.88 |
| (Panalane H-300) | | | |
| Styrene-isoprene-styrene block | 20.00 | 20.00 | 20.00 |
| copolymer | | | |
| Mineral oil | 15.00 | 15.00 | 15.00 |
| Isopropyl myristate | 0.00 | 5.00 | 10.00 |
| Silicon dioxide | 1.00 | 1.00 | 1.00 |
| Lidocaine | 1.80 | 1.80 | 1.80 |

### Manufacturing Process:

### Process:

1. Dispensing Toluene: Begin by dispensing toluene into the mixing jar.
2. Adding Ingredients: Add colloidal silicon dioxide, mineral oil, IPM, and lidocaine into the mixing tank. Mix thoroughly to dissolve the lidocaine and disperse the silicon dioxide.
3. Incorporating SIS Polymer: Add the SIS polymer and mix until it is fully solvated.
4. Adding Oppanol and Panalane: Follow by adding Oppanol high molecular weight and mix until it is solvated, then add Oppanol B12 and Panalane H-300, mixing until a homogeneous mixture is achieved.

The solid content of this mixture is 36%. The solid content of the drug in adhesive (DIA) is critical for achieving the desired coat weight.
5. Coating and Drying: The DIA is then coated onto a PET film and dried to reach a target coat weight of 7.14 mg/cm². The resulting laminate is then laminated with a non-woven fabric.
6. Second Layer Application: A second layer of DIA is coated onto the PET liner to achieve the same target coat weight of 7.14 mg/cm². As this second layer exits the drying tunnel, it is laminated with the previously coated non-woven fabric, effectively sandwiching the adhesive layer between the non-woven fabric and the PET release liner.

### IVPT Performance Summary:

In-vitro permeation studies revealed the following results:

| IPM/MO | Cumulative Amt. | Average Flux | Max. Flux | Min. Flux |
|---|---|---|---|---|
| (% w/w) | (μg/cm²) | (μg/cm²/h) | (μg/cm²/h) | (μg/cm²/h) |
| RLD | 69.21 ± 16.65 | 2.69 | 3.76 | 0.89 |
| 5/15 (Target) | 85.50 ± 5.16 | 3.32 | 4.68 | 0.53 |
| 0/15 | 75.38 ± 17.67 | 2.95 | 4.14 | 0.71 |
| 10/15 | 88.51 ± 14.41 | 3.32 | 4.23 | 0.36 |
| 5/10 | 77.53 ± 17.62 | 2.74 | 4.26 | 0.49 |
| 5/20 | 89.64 ± 18.42 | 3.51 | 4.50 | 0.60 |

From the results observed across the formulation examples, several important conclusions can be drawn regarding the optimization of a lidocaine-containing non-aqueous patch. The experimental data demonstrate that the balance between mineral oil (MO) and isopropyl myristate (IPM) plays a pivotal role in modulating the solubility, thermodynamic activity, and subsequent drug release of lidocaine from the adhesive matrix. It was consistently observed that lidocaine exhibits high solubility in IPM and very limited solubility in mineral oil. When the proportion of mineral oil was increased in the formulation, the solubility of lidocaine decreased, leading to a partially solubilized state. This reduced solubility increased the thermodynamic activity of lidocaine, resulting in enhanced drug release profiles as evidenced by in-vitro permeation testing (IVPT). Specifically, formulations containing 15% w/w mineral oil exhibited a marked improvement in drug flux compared to those containing only 10% w/w mineral oil. Conversely, increasing IPM beyond 5% w/w did not yield proportionate improvements in drug release, indicating a plateau effect, with optimal flux observed at approximately 5% IPM.

Furthermore, crystallization studies revealed that lidocaine remained stable in a non-crystalline state up to 1.8% w/w concentration under both real-time and accelerated storage conditions. Crystallization tendencies were only observed at the highest tested concentration of 5% w/w lidocaine, primarily under accelerated conditions, suggesting the selected range of 1.8% w/w ensures superior physical stability during storage and shelf-life. The combination of Oppanol N80 or N100 and Oppanol B12 grades of polyisobutene, along with Panalane H-300 and SIS block copolymer, contributed to maintaining adhesive integrity while facilitating controlled drug release.

The inclusion of mineral oil and IPM in specific ratios not only controlled drug solubility but also optimized mechanical and adhesive properties of the patch. Increasing mineral oil beyond 15% w/w negatively impacted adhesion, whereas lower concentrations reduced drug release. The optimized formulation thus ensures adequate adhesion alongside high thermodynamic activity for effective drug permeation. The in-vitro permeation studies further validated these findings, with the optimized formulation demonstrating higher drug release than the reference product, ensuring both therapeutic effectiveness and patient compliance. Collectively, these results confirm that the controlled adjustment of mineral oil and IPM concentrations allows for a finely tuned balance between drug solubility, release kinetics, adhesive performance, and storage stability, making the formulation suitable for safe, sustained delivery of lidocaine through a non-aqueous patch system.

The present invention as described herein discloses a lidocaine non-aqueous patch comprising from about 0.1% to about 7% by weight of lidocaine in a non-aqueous adhesive matrix composed of high molecular weight polyisobutylene (HMW PIB), low molecular weight polyisobutylene (LMW PIB), polybutene, styrene-isoprene-styrene (SIS) block copolymer, mineral oil, silicon dioxide, and isopropyl myristate. The lidocaine concentration may range from about 0.5% to about 3%, with preferred concentrations between about 1.0% and about 2.0%, and a more preferred concentration of about 1.8% by weight. The high molecular weight polyisobutylene is typically present in an amount from about 5% to about 20%, preferably from about 8% to about 15%, and the low molecular weight polyisobutylene may range from about 15% to about 40%, preferably from about 20% to about 35%. Polybutene may be included at a concentration from about 15% to about 35%, more preferably from about 20% to about 30%. The SIS block copolymer may be present from about 10% to about 30%, more preferably from about 20% to about 25%. Mineral oil may be used in amounts from about 5% to about 20%, more preferably from about 10% to about 15%, and isopropyl myristate may range from about 1% to about 15%, more preferably from about 5% to about 10%. The ratio of isopropyl myristate to mineral oil may range between 1:1 to 4:1, more preferably between 1:2 to 1:3. The formulation may optionally include additional excipients such as zinc oxide, zinc stearate, butylated hydroxytoluene (BHT), or other antioxidants and stabilizers. The patch may contain lidocaine loading in the range of about 0.1 mg/cm² to about 5 mg/cm², preferably about 0.1 mg/cm² to about 1 mg/cm², with particular preference for about 0.5 mg/cm² to about 0.8 mg/cm². The formulation may be prepared by a solvent coating process wherein all ingredients are dissolved or dispersed in an organic solvent selected from toluene, heptane, or ethanol, followed by coating and drying. Toluene is a preferred solvent due to its favorable effect on the adhesive balance. The adhesive matrix may be coated onto a polyethylene terephthalate (PET) backing at a coat weight from about 7 mg/cm² to about 20 mg/cm², preferably between 10 mg/cm² and 15 mg/cm², with a particularly preferred target of 14.28 mg/cm². A multilayer laminate structure may be utilized where the adhesive layer is sandwiched between a PET liner and a non-woven fabric layer. The formulation may be stable without crystallization of lidocaine when stored under ICH long-term and accelerated conditions for at least three to six months. The patch may release at least 5% of lidocaine after 12 hours, preferably greater than 6% and up to 10%. In-vitro permeation rates may achieve a flux of at least 2.5 μg/cm²/hour, more preferably between 3.0 to 4.0 μg/cm²/hour, measured by Franz cell diffusion methodology. The patch mass per unit area may range from about 60 g/m² to about 200 g/m², preferably from about 135 g/m² to 150 g/m². The backing layer may comprise biaxially-oriented stretch cloth having peel strength between 0.4 N/25 mm and 5 N/25 mm, preferably between 1 N/25 mm to 3 N/25 mm as determined by 180-degree peel test. The patch may be packaged in a moisture-resistant, light-protective pouch comprising polyethylene or a polyethylene-aluminum laminate. The disclosure further includes a method for treating pain by applying the lidocaine patch to an affected area, including conditions such as post-herpetic neuralgia, localized muscle or joint pain, and diabetic neuropathy. The method provides pain relief lasting at least 12 to 24 hours while minimizing skin irritation. The formulation may be substantially free of water (less than 1% by weight) and free of alcohol-based solvents, minimizing the risk of irritation. Enhanced thermodynamic activity of lidocaine may be achieved through a partially solubilized state within the matrix, supporting improved drug delivery. Optional use of alternative solubilizers or permeation enhancers such as oleic acid or stearic acid may also be included. The formulation may achieve sustained dermal lidocaine levels suitable for extended therapeutic application with improved adhesion and patient comfort.

As will be apparent to those skilled in the art to which the invention pertains, the present invention may be embodied in forms other than those specifically disclosed above without departing from the spirit or essential characteristics of the invention. The particular embodiments of the invention described above, are, therefore, to be considered as illustrative and not restrictive. The scope of the present invention is as set forth in the appended claims rather than being limited to the examples contained in the foregoing description.

What we claim is:

1. A lidocaine non-aqueous patch, comprising:
about 1.8% w/w lidocaine in a non-aqueous base,
high molecular weight polyisobutylene,
low molecular weight polyisobutylene,
polybutene,
styrene/isoprene/styrene block copolymer,
mineral oil,
silicon dioxide, and
isopropyl myristate.

2. The lidocaine non-aqueous patch of claim 1, wherein the release ratio of lidocaine from the patch is greater than 6% w/w after 12 hours of attachment to the skin.

3. The lidocaine non-aqueous patch of claim 1, wherein the amount of lidocaine is between 0.1 mg/cm² and 1 mg/cm² of the patch.

4. The lidocaine non-aqueous patch of claim 1, wherein the concentration of styrene/isoprene/styrene block copolymer is between 20% w/w and 25% w/w.

5. The lidocaine non-aqueous patch of claim 1, wherein the concentration of isopropyl myristate is between 5% w/w and 10% w/w.

6. The lidocaine non-aqueous patch of claim 1, wherein the concentration of mineral oil is between 5% w/w and 15% w/w.

7. The lidocaine non-aqueous patch of claim 1, wherein the ratio of isopropyl myristate to mineral oil is between 1:2 and 1:3 by weight.

8. The lidocaine non-aqueous patch of claim 1, further comprising zinc oxide, zinc stearate, and an antioxidant.

9. The lidocaine non-aqueous patch of claim 1, wherein the patch is prepared using a solvent coating process comprising the steps of mixing all excipients in an organic solvent, followed by coating and drying to achieve a desired coat weight.

10. The lidocaine non-aqueous patch of claim 9, wherein the solvent is toluene.

11. The lidocaine non-aqueous patch of claim 9, wherein the patch is prepared by placing styrene/isoprene/styrene block copolymer, polyisobutylene, polybutene, silicon dioxide, butylated hydroxytoluene, mineral oil, isopropyl myristate, and lidocaine into toluene, and the resulting solution is coated onto a polyester film to achieve a desired coat weight.

12. The lidocaine non-aqueous patch of claim 9, wherein the patch is prepared by dispensing toluene into a mixing vessel, adding colloidal silicon dioxide, mineral oil, isopropyl myristate, and lidocaine, and stirring until the lidocaine is dissolved and the silicon dioxide is dispersed; then adding styrene/isoprene/styrene block copolymer and stirring until solvated; followed by sequential addition of high molecular weight and low molecular weight polyisobutylene and polybutene, and mixing until a homogeneous mixture is achieved.

13. The lidocaine non-aqueous patch of claim 12, wherein the homogeneous drug-in-adhesive mixture is coated onto a polyethylene terephthalate (PET) film and dried to achieve a target coat weight, and the dried laminate is laminated with a non-woven fabric.

14. The lidocaine non-aqueous patch of claim 13, wherein a second layer of the drug-in-adhesive mixture is coated onto a PET liner to achieve the target coat weight, and the second layer is laminated with the previously coated non-woven fabric, sandwiching the adhesive layer between the non-woven fabric and the PET release liner.

15. The lidocaine non-aqueous patch of claim 13, wherein the target coat weight is about 14.28 mg/cm².

16. The lidocaine non-aqueous patch of claim 1, wherein the formulation is free of lidocaine crystallization under storage conditions for at least four weeks.

17. The lidocaine non-aqueous patch of claim 1, wherein the patch exhibits physical and chemical stability under at least one of the following storage conditions: refrigerated (4° C.±2° C.), controlled room temperature (25° C.±2° C., 60% RH±5% RH), or accelerated conditions (40° C.±2° C., 75% RH±5% RH) for at least four weeks without lidocaine crystallization or significant degradation of lidocaine.

18. The lidocaine non-aqueous patch of claim 1, wherein the patch provides a lidocaine flux of between 2.5 μg/cm²/hour to 3.5 μg/cm²/hour over a 12-hour period as determined by in-vitro permeation testing using Franz diffusion cells and human cadaver skin.

19. A method of treating pain associated with post-herpetic neuralgia, comprising applying the lidocaine non-aqueous patch of claim 1 to an affected area of skin, thereby providing pain relief without causing significant skin irritation.

20. A packaged pharmaceutical product comprising the lidocaine non-aqueous patch of claim 1 enclosed within a moisture-impermeable pouch.
